(19) United States
(12) Patent Application Publication
(10) Pub. No.: US 2019/0388009 A1
(43) Pub. Date: Dec. 26, 2019

(54) ASSESSING NERVE CONDUCTION IN RESPONSE TO AN ORAL CARE COMPOSITION

(71) Applicant: The Procter & Gamble Company, Cincinnati, OH (US)

(72) Inventors: Ying TANG, Beijing (CN); Ying XU, Singapore (SG)

(21) Appl. No.: 16/445,763

(22) Filed: Jun. 19, 2019

(30) Foreign Application Priority Data
Jun. 22, 2018 (CN) CN2018/092341

(51) Int. Cl. A61B 5/11; A61N 1/05; A61B 5/00

(52) U.S. Cl. CPC A61B 5/1106; G09B 23/283; A61B 5/4041; A61N 1/0548

(57) ABSTRACT

A method of assessing nerve conduction of a trigeminal nerve in a subject in response to an oral cavity treatment by an oral composition helps improve/design oral care compositions, especially those having a sensate.

### FIELD OF THE INVENTION

The present invention relates to an objective method for assessing oral care compositions in the oral cavity of a subject.

### BACKGROUND OF THE INVENTION

Oral care compositions (e.g., dentifrice compositions) are well known for dental and oral hygiene care. The use of sensates (warming agents, cooling agents, etc.) are well known. Designing or formulating oral care composition is typically done with expert human sensory panels, wherein the expert human panelists assess sensory of the oral care compositions by giving ratings. Although many of these panels are highly trained and calibrated, there is an opportunity for even greater objectivity when assessing oral compositions, especially those oral care compositions comprising sensates.

### SUMMARY OF THE INVENTION

The present invention address this opportunity by providing, in one aspect of the invention, a method of assessing nerve conduction of a trigeminal nerve in a subject in response to an oral cavity treatment by an oral composition comprising the steps: (a) treating the subject's oral cavity with the oral care composition; (b) affixing a probe in or adjacent the subject's oral cavity so the probe is in electrical communication with the subject's trigeminal nerve; and (c) assessing nerve conduction of the subject trigeminal nerve via the affixed probe.

Another aspect of the invention provides for a kit for providing a product demonstration comprising: (a) an electrical current perception threshold (CPT) evaluating device; (b) a probe functionally connectable to said device; (c) the product comprises an oral care composition; and (d) optionally use instructions. Methods of using the kit for a product demonstrate is also provided.

While the specification concludes with claims that particularly point out and distinctly claim the invention, it is believed the present invention will be better understood from the following description.

### DETAILED DESCRIPTION OF THE INVENTION

### Definitions

The term “effective amount” as used herein means an amount of a compound or composition sufficient to induce a positive benefit, an oral health benefit, and/or an amount low enough to avoid serious side effects, i.e., to provide a reasonable benefit to risk ratio, within the sound judgment of a skilled artisan. In one example, “effective amount” means at least 0.001% of the material, by weight of the composition, alternatively at least 0.1%.

The term “oral care composition,” as used herein, is meant a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces. One example of an oral care composition is a dentifrice.

The term “sensates” are sensate molecules, such as cooling, warming, and tingling agents, useful to deliver signals to the user. Sensates may also be part of an oral care composition. Sensates are generally present in an amount of from about 0.001% to about 0.8%, by weight of the oral care composition. For example, a known cooling agent is menthol. Menthol provides this sensation by acting as a TRPM8 agonist. In another example, a known warming agent is vanillyl butyl ether (“VBE”). VBE provides this warming sensation by acting as a TRPV1 agonist.

The term “co-sensate” is a molecule that influences the effect of a sensate. One example of a co-sensate is certain diols.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt %” herein. All molecular weights as used herein are weight average molecular weights expressed as grams/mole, unless otherwise specified.

As used herein, the articles including “a” and “an” when used in a claim, are understood to mean one or more of what is claimed or described.

As used herein, the terms “comprise”, “comprises”, “comprising”, “include”, “includes”, “including”, “contain”, “contains”, and “containing” are meant to be non-limiting, i.e., other steps and other sections which do not affect the end of result can be added. The above terms encompass the terms “consisting of” and “consisting essentially of”.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

One aspect of the invention provides for a method of assessing nerve conduction of a trigeminal nerve in a subject in response to an oral cavity treatment by an oral composition comprising the steps: (a) treating the subject oral cavity with the oral care composition; (b) affixing a probe in or adjacent the subject's oral cavity so the probe is in electrical communication with the subject's trigeminal nerve; and (c) assessing nerve conduction (1 unit=0.01 milli-ampere) of the subject trigeminal nerve via the probe.

Preferably the oral care composition comprises a sensate, preferably wherein the sensate is selected from a cooling agent, warming agent, tingling agent, or combinations thereof. Preferably the oral care treatment comprises brushing teeth with the oral care composition, wherein the oral care compositions is preferably a toothpaste.

Preferably the conduction is assessed from 1 Hz to 20 Hz, preferably from 2 Hz to 10 Hz, more preferably from 3 Hz to 7 Hz, alternatively about 5 Hz. Alternatively, the conduction is assessed from 200 Hz to 300 Hz, preferably from 225 Hz to 275 Hz, alternatively about 250 Hz. Preferably the probe is attached either on the gum or mucosa of the oral cavity, or the non-mucosa skin adjacent the cavity. Preferably the probe is in electrical communication to the mandibular branch of the trigeminal nerve. Preferably the method further comprising the step of correlating a user's pain threshold based upon the assessed conduction.

The method may include establishing a base-line or control by providing additional steps of affixing a probe in or adjacent the subject's oral cavity so the probe is in electrical communication with the subject's trigeminal nerve; and assessing nerve conduction (1 unit=0.01 milli-ampere) of the subject trigeminal nerve via the probe, such that these additional steps are conducted before treating the subject oral cavity with the oral care composition. The method may also include steps to assess results more than one time over the course of a time frame. For example, the method comprises conducting said affixing and assessing steps in a time frame from immediately after oral care treatment to 30 days after oral care treatment, preferably said affixing and assessing steps are repeated (2, 3, or more times) within this time frame.

### EXAMPLES

Data is provided to demonstrate the impact of having diols as a co-sensate in the dentifrice composition to help mitigate against lowering of the pain threshold in the oral cavity that otherwise happens in the presence of VBE (i.e., a TRPV1 agonist). An electrical current perception threshold (CPT) evaluating device is used to assess the trigeminal nerve at 5 Hertz (Hz) and 250 Hz in oral care compositions with and without diols. Results are reported as milliamps (1 unit=0.01milli-ampere). Using microprocessor controlled, neuroselective electrical stimuli, the instrument quickly quantifies the conduction and functional integrity of the large and small myelinated and small unmyelinated sensory nerve fibers at any cutaneous site. Alternatively, the mucosal or gum area may also be assessed (but not assessed in this study). The device used is the a Neurometer® CPT®/C neuroselective sensory Nerve Conduction Threshold (sNCT™) electrodiagnostic testing device, from Neurotron, Incorporated (Denver, Colo., USA). See Operating Manual, Version18.3, copyright 2010; and www.neutron.com.

Electrical CPT evaluation quantifies the sensory threshold to transcutaneous electrical stimulation of the sensory nerves. 5 Hz is the frequency for C fiber which transmits pain and warm. 250 Hz is the frequency for A delta fiber which transmits fast pain. The data herein shows a significant correlation between CPT and skin sensation. It is known that sensations (e.g., cooling, heat, pressure, pain, tingling, and the like) from mouth are transmitted into brain through the trigeminal nerve. Therefore, CPT measurement at, for example, the mandibular division of this nerve is a way to objectively understand how users of oral care compositions respond to different sensates.

Two groups of 10-15 subjects in each group are provided. Subjects are randomized for gender and age. The test is conducted, in the relevant portion, over five days. Subjects are preconditioned before the test. Members of the first group brush their teeth with Example A toothpaste (in Table 3 below) and the second group with Example B toothpaste. The notable difference between these two compositions is the presence of diols. That is, example B contains diols whereas example A does not. Although both compositions have 0.45 wt % of menthol, the point of this study is to measure how diols may reduce pain, i.e., decrease the reduction of the pain threshold (vs. e.g., assessing the sensorial experience).

TABLE 1
Ingredients and Weight Percentages (Wt %) of Toothpaste
Compositions Examples A and B are provided.

| | Example A | Example B |
|---|---|---|
| Ingredient: | (Wt %) | (Wt %) |
| 1-Menthol | 0.45 | 0.45 |
| TRPV1 agonist | 0.04 of VBE | 0.1 of |
| | (nil diols) | THERMOLAT ®³ |
| | | (0.04 of VBE) |
| Sorbitol solution | 66 | 66 |
| (70 wt % active) | | |
| Water added (treated) | q.s | q.s |
| Sodium fluoride | 0.243 | 0.243 |
| monobasic sodium phosphate | 0.4 | 0.4 |
| Sodium phosphate | 0.93 | 0.93 |
| tribasic dodecahydrate | | |
| Saccharin sodium | 0.30 | 0.30 |
| Carboxymethyl cellulose | 0.7 | 0.7 |
| Carbomer | 0.22 | 0.22 |
| Silica (ZEODENT ®) | 16 | 16 |
| Sodium lauryl sulfate | 7.0 | 7.0 |
| (29 wt % active) | | |
| Flavor - citrus mint | 0.55 | 0.55 |
| (excluding menthol) | | |
| Target Viscosity (BKU⁴) | 8-37 | 8-37 |
| Target pH | 6.5-7.5 | 6.5-7.5 |

³THERMOLAT ® is from Symrise, product number 793238. The commercial literature from Symrise (dated 2017) reports 1 wt % of THERMOLAT ® has 0.4% vanillyl butyl ether (i.e., “VBE” as the TRPV1 agonist) wherein the remaining amount has unspecified levels of the diols 1,2-hexandiol, caprylyl glycol (i.e., 1,2-octanediol), and antioxidant ascorbyl palmitate.
⁴1 BKU = 10,000 centipoise

The following steps are taken for each of the five days of the study. The CPT assessment is undertaken at Day 1 and then again at Day 5. Specifically, the assessment is taken before brushing and after brushing on the first day. Lastly, the assessment is taken at the fifth day. At least one hour before each assessment, there is no eating, drinking, chewing gum, or toothbrushing (so as not to interfere with the results). The treatment or test for each day is conducted in a clinical laboratory.

TABLE 2
Summary of the CPT test method over five days.

| Day: | Treatment or Test: |
|---|---|
| 1 | Step 1: CPT test at the mandibular division |
| | of the trigeminal nerve (“Jaw”). |
| | Step 2: Brush teeth with composition C or D. |
| | Step 3: CPT test at Jaw. |
| 2 | Use subject toothpaste. |
| 3 | Use subject toothpaste. |
| 4 | Use subject toothpaste. |
| 5 | CPT test at Jaw. |

Results for the two groups (using toothpaste example A or B, respectively) are provided in Table 3A. Results are reported as milliamps wherein 1 unit is equal to 0.01 milli-ampere and provided at the 5 Hz and 250 Hz as well as an averaging of the two frequencies. A ‘value’, to determine whether there are any statistically significant differences, are provided in Table 3B below.

TABLE 3A
Least Square means at 5 Hz/250 Hz/5 + 250 Hz

| | | Least Square Means (Standard Error) | | |
|---|---|---|---|---|
| | | Day 1 Before | Day 1 After | Day 5 Before |
| Ex.: | Hz | Brushing | Brushing | Brushing |
| A | 5 | 14.5 (2.4) | 7.7 (1.7) | 13.7 (1.8) |
| B | 5 | 12.9 (2.4) | 12.2 (1.7) | 14.7 (1.8) |
| A | 250 | 26.1 (3.5) | 17.6 (2.9) | 30.1 (3.3) |
| B | 250 | 26.7 (3.5) | 26.4 (2.9) | 30.2 (3.3) |
| A | 5 + 250 | 39.8 (5.5) | 25.1 (4.2) | 43.4 (4.6) |
| B | 5 + 250 | 39.8 (5.5) | 38.3 (4.2) | 45.3 (4.6) |

TABLE 3B
P values with respect to Table 3A

| | | P value | | |
|---|---|---|---|---|
| | | Day 1 Before | Day 1 Before | Day 1 After |
| | | Brushing vs. | Brushing vs. | Brushing vs. |
| | | Day 1-After | Day 5 Before | Day 5 Before |
| Ex.: | Hz | Brushing | Brushing | Brushing |
| A | 5 | 0.0004 | 0.7569 | 0.0245 |
| | | (Significant) | | (Significant) |
| B | 5 | 0.6267 | 0.4841 | 0.3131 |
| A | 250 | 0.003 | 0.4144 | 0.0037 |
| | | (Significant) | | (Significant) |
| B | 250 | 0.8885 | 0.4852 | 0.3337 |
| A | 5 + 250 | 0.0006 | 0.6131 | 0.0054 |
| | | (Significant) | | (Significant) |
| B | 5 + 250 | 0.6856 | 0.4427 | 0.2529 |

Results of Table 3A and 3B are discussed. An acute response of the diols is provided in Day 1 results. A cumulative response is provided by the diols when comparing between Day 1 and Day 5 results at 5 Hz. There is a statistically significant difference discovered on Day 1, before and after brushing, when subjects use toothpaste composition A (which does not contain diols). In contrast, there is no difference assessed on Day 1 for those users using toothpaste composition B (which contains diols). The difference between these compositions is the presence of diols. The presence of diols in Example B suggests the pain threshold does not decrease in the presence of diols in contrast to when the diols are not present (and the pain threshold decreases). Without wishing to be bound by theory, this decrease in the pain threshold may make the user more sensitive to irritants in the oral cavity.

A cumulative response of the diols is provided when comparing Day 1 after brushing and Day 5 before brushing. That is, there is a statistically significant difference discovered when subjects use toothpaste A at this time-point, in contrast to comparing to those subjects using toothpaste B. Net, the presence of diols in example B suggests the pain threshold does not decrease over this time-period as compared to when the diols are not present (and the pain threshold decreases).

Analogous results are observed when the study is conducted at 250 Hz and when averaging the results of 5 Hz and 250 Hz. Thus, the presence of diols can help provide dentifrice compositions, of the present invention, which not only provides the desired dominant heating and subsequent dominant cooling benefits, but also provides the user the additional benefit of helping to keep the pain threshold from decreasing (thereby helping the user not become more susceptible to irritants in the oral cavity).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of assessing nerve conduction of a trigeminal nerve in a subject in response to an oral cavity treatment by an oral composition comprising the steps:
(a) treating the subject oral cavity with the oral care composition;
(b) affixing a probe in or adjacent the subject's oral cavity so the probe is in electrical communication with the subject's trigeminal nerve; and
(c) assessing nerve conduction of the subject trigeminal nerve via the affixed probe.

2. The method of claim 1, wherein the oral care composition comprises a sensate, preferably wherein the sensate is selected from a cooling agent, warming agent, tingling agent, or combinations thereof.

3. The method of claim 1, wherein the conduction is assessed from 1 Hz to 20 Hz, preferably from 2 Hz to 10 Hz, more preferably from 3 Hz to 7 Hz, alternatively about 5 Hz.

4. The method of claim 1, wherein probe is attached either on the gum or mucosa of the oral cavity, or the non-mucosa skin adjacent the cavity.

5. The method of claim 1, wherein probe is in electrical communication to the mandibular branch of the trigeminal nerve.

6. The method of claim 1, wherein oral care treatment comprises brushing teeth with the oral care composition, wherein the oral care composition is a toothpaste.

7. The method of claim 1, further comprising the step of correlating a user's pain threshold based upon the assessed conduction.

8. The method of claim 1, wherein the oral care composition further comprises a co-sensate.

9. The method of claim 1, further comprising the additional steps of affixing a probe in or adjacent the subject's oral cavity so the probe is in electrical communication with the subject's trigeminal nerve; and assessing nerve conduction of the subject trigeminal nerve via the probe;
wherein said additional steps are conducted before treating the subject oral cavity with the oral care composition (to establish a baseline).

10. The method of claim 1, wherein said affixing and assessing steps are conducted are conducted in a time frame from immediately after oral care treatment to 30 days after oral care treatment, more preferably said affixing and assessing steps are repeated (2, 3, or more times) within this time frame.

11. A kit for providing a product demonstration comprising:
(a) an electrical current perception threshold (CPT) evaluating device;
(b) a probe functionally connectable to said device;
(c) the product comprises an oral care composition;
(d) optionally use instructions.

12. The kit of claim 11, wherein the oral care composition is a toothpaste composition, and wherein the toothpaste composition comprises a sensate.
